(19) United States
(12) Patent Application Publication
(10) Pub. No.: US 2016/0008502 A1
(43) Pub. Date: Jan. 14, 2016

(54) System and Apparatus for Disease Prevention

(71) Applicant: Liada LaBonte, Seminole, FL (US)

(72) Inventor: Liada LaBonte, Seminole, FL (US)

(21) Appl. No.: 14/325,660

(22) Filed: Jul. 8, 2014

(51) Int. Cl. A61L 2/22; A47K 5/00

(52) U.S. Cl. CPC A61L 2/22; A47K 5/00

(57) ABSTRACT

A hand sanitizer dispenser includes a body portion having a reservoir for holding a liquid. A first end of the body portion has a device for dispensing the liquid. A second end of the body portion (the second end opposes the first end) has a hook portion either formed or affixed to the second end of the body portion.

### FIELD

This invention relates to the field of disease prevention and more particularly to a system for isolating hands from germs while providing a source of antibacterial spray.

### BACKGROUND

Some people have become rightfully cautious of exposure to microbes or microorganisms (bacteria, viruses, fungi, spores, etc.). Although, at times taken too far, as for example, bringing one's own plastic dining utensils to a restaurant, tests have shown certain places and objects as being virtual petri dishes of microorganisms. For example, gyms, schools, offices, etc., often breed and transfer such microbes to people. The simple act of opening the door is capable of transferring microbes from the door handle to a person's hands. While not necessarily a danger in itself, the microbes typically remain active on the person's hands for hours and, later, a touch of an eye, an open wound, or contact with the person's mouth allows some of those microbes access to a warm, moist part of the body, where the microbes are free to live and multiply, causing illness and, unfortunately for some, death. Furthermore, the affected person then transfers microbes to other people who transfer the microbes to other still other people, and so on.

In recent years, new, highly contagious diseases such as necrotizing fasciitis and Methicillin-resistant Staphylococcus Aureus (MRSA) have surfaced, believed to be mutations of previous viruses that were treated by partial doses of antibiotics. These viruses mutated in such a way as to become resistant to the same antibiotics that were originally used to treat their ancestors. By being resistant to antibiotics, it is very difficult to treat patients who are infected by these viruses and, therefore, unless the person's own immune system is successful, death is a possible outcome. Recently, yet another virus called Middle east respiratory syndrome coronavirus (MERS) has spread from where it is believed to have originated in the Mideast to many countries. Often, an affected individual traveling back from such an area carries the virus, affecting many others during their travel and still more after arrival at their destination.

Some microbes are spread in the air and, after inhaling, the microbes find suitable growth environments in peoples lungs. Some microbes are transmitted by contact, in that, the microbe does not move on its own. Rather, the microbe is deposited by an affected person onto an object and another person, touching that object, acquires the microbe. After acquisition, if the microbe is later transferred to an area of the person that is susceptible to such invasion (eyes, mouth, nose, open wound, etc.), that person often becomes affected. This is where washing of one's hands is important, in that, if, after the person acquires the microbe, the person correctly washes their hands, the microbe is washed away and the person does not become affected.

Unfortunately, it is often very difficult to wash one's hands after they touch anything. Even the process of going into a rest room to wash your hands requires exiting that rest room and, more than likely, touching the door or door knob after washing. Those with serious concerns over microbes often perform various acts when exiting a rest room such as opening the door with their feet, saving the paper towel used to dry their hands to isolate the door knob from their hands, using a part of their clothing to open the door knob, waiting for someone else to open the door, etc.

Instead of washing hands, the use of a germicidal fluid has become popular. Often called antibacterial hand sanitizers, these fluids are often provided in bottles for dispensing or spraying onto a person's hands. Such fluids are often alcohol-based and are effective at neutralizing or reducing the spread of most microbes. Some such hand sanitizers include moisturizers, Aloe, vitamins, fragrances, coloring, etc. to make their use more appealing. Unfortunately, the containers that hold these antibacterial fluids are often not readily accessible when needed. For example, after a woman touches a door knob when leaving a rest room, that woman searches through her handbag to find her little bottle of hand sanitizer. Spraying the hand sanitizer on her hands kills most of the microbes that remain on her hands, but some of the microbes have been deposited on her handbag and other contents and can be transferred back to the woman upon later access of the handbag. Likewise, if a man does the same and accesses a small bottle of sanitizer in his pocket, some of the microbes are transferred to his pocket and other contents for later transmittal back to the man.

What is needed is a system and apparatus that will provide a convenient dispenser for a sanitizer while also providing a tool for a user to open objects without directly touching the objects.

### SUMMARY

In one embodiment, a hand sanitizer dispenser is disclosed including a body portion having a reservoir for holding a liquid. A first end of the body portion has a way to dispense the liquid. A second end of the body portion (the second end opposes the first end) has a hook portion either formed or affixed to the second end of the body portion.

In another embodiment, a method of maintaining hand sanitation is disclosed including providing a hand sanitizer dispenser with hook. The hand sanitizer dispenser has a body portion including a reservoir for holding a liquid. A first end of the body portion has a way to dispense the liquid. A second end of the body portion (the end opposing the first end) has a hook portion. The method includes holding the body portion in one hand and engaging an object with the hook portion to move the object in a desired direction. The object is moved by way of the hook portion without making direct contact between the hand and the object. After moving the object to a desired position, the object is released from the hook portion.

In another embodiment, a hand sanitizer dispenser is disclosed including a body portion that has a reservoir for holding a liquid. A first end of the body portion has a sprayer for dispensing the liquid and a second end opposing the first of the body portion has a hook portion that is made of a stiff material. A cover, the cover removably engaged to the first end of the body for covering the sprayer.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a perspective view of a hand sanitizer dispenser of the prior art.

FIG. 2 illustrates a perspective view of a hand sanitizer dispenser with a handle engagement mechanism affixed.

FIG. 3 illustrates a perspective view of a hand sanitizer dispenser with the handle engagement mechanism removably attached.

FIG. 4 illustrates a perspective view of a hand sanitizer dispenser with the handle engagement mechanism integrated.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

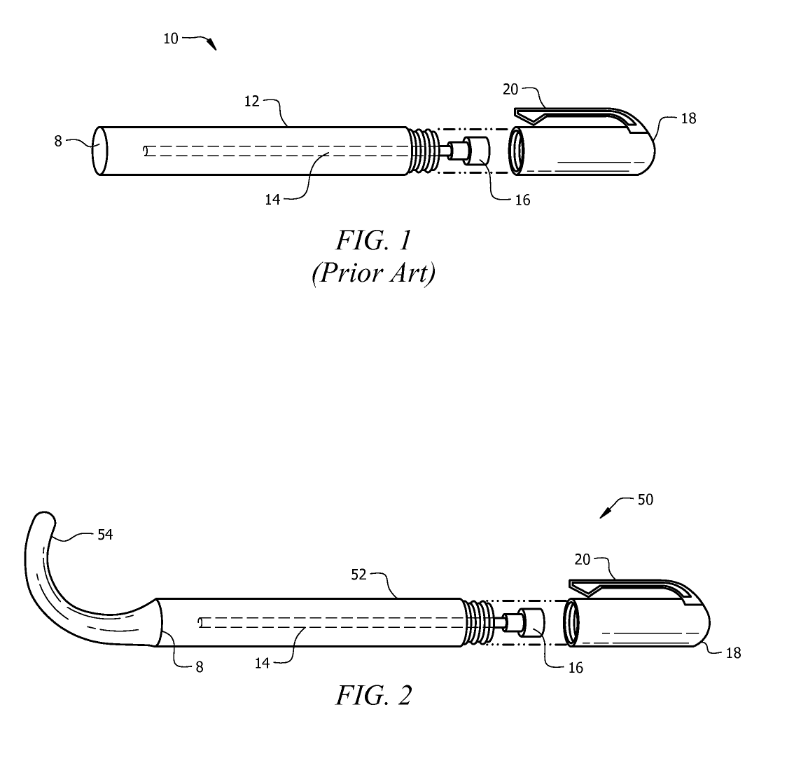

Referring to FIG. 1, a perspective view of a hand sanitizer dispenser 10 of the prior art is shown. There are many varieties and shapes of hand sanitizer dispensers 10, some atomizing the hand sanitizer solution and some simply dispensing or pouring amounts of the hand sanitizing solution onto surfaces and one's skin. In the example of such shown in FIG. 1, the hand sanitizer dispenser 10 has a body portion 8/12/14/16 and a cap or cover portion 18/20. The body portion 8/12/14/16 has a generally hollow, often tubular reservoir 12 for holding a liquid (the hand sanitizer fluid), a bottom 8 that closes the reservoir 12, and a dispensing portion 14/16 that includes a feed tube 14. When the hand sanitizer dispenser 10 is held upright (the bottom 8 is downward towards earth), the hand sanitizer fluid settles near the bottom 8 and, when the spray 16 is operated, the hand sanitizer fluid is drawn up the feed tube 14 and sprayed/atomized into the air around where the spray 16 is aimed.

To reduce leakage and inadvertent activation of the spray 16, many such hand sanitizer dispensers 10 include a cover 18. Also, many covers 18 have a clip 20 for removably affixing the hand sanitizer dispenser 10 to an article of clothing, a handbag, etc.

Referring to FIGS. 2, 3, and 4, perspective views of hand sanitizer dispensers 50/60/70 with handle engagement mechanism are shown. As described previously, one way for a person to acquire microbes is by touch. There are often active microbes hiding on many surfaces that a person typically comes into contact with, after all, that is likely where the microbes came from in the first place—another person touching the same surface. Some common surfaces that require a touch by a person are elevator buttons, door knobs, trash can lids, faucets, copy machine buttons, light switches, airplane door pulls, grocery bags, and many, many other surfaces. There are several ways of reducing the risk of acquiring microbes from many of these surfaces, including, not touching the surfaces (e.g. wait for someone else to press the elevator button and say, “17 please”), isolating your body from the surfaces with clothing, paper towels, etc., washing your hands after contacting the surfaces, using a sanitizer after contacting the surfaces, etc.

As previously described, the later scenario (using a sanitizer) has been widely practiced by carrying a dispenser 10 of sanitizer such as that shown in FIG. 1 of the prior art. Likewise, it was previously described that there are issues with doing such, in that, after touching a surface with one's hands, microbes that the hands acquire from the surface readily transfer from the hands onto other surfaces such as handbags, contents of the handbags, clothing, contents of pockets, etc. In some cases, the microbes transfer onto surfaces of the dispenser 10, itself. Later, while the microbes are still viable, if that person touches one of these other surfaces, they reacquire the microbes and are then capable of infecting their body with the microbes by, for example, touching their mouth, nose, eyes, an open wound, etc.

People have found ways to avoid touching some surfaces, thus limiting acquisition of many microbes, by, for example, waiting for someone else to touch the surface for them, etc. There are still, many surfaces that require a specific type of action which is very difficult to perform using a bypass scenario, for example, those described above. Consider a person in a public restroom. There are no paper towels, only hand blowers. The doors open inward and there is a door handle. It is late and it is unlikely that another will enter the restroom. What is this person to do? They could use part of their clothing to isolate themselves from the door handle, but then their clothing would acquire the microbes for possible later transmittal into the person's body.

The hand sanitizer dispensers 50/60/70 disclosed in FIGS. 2-4 provide a hooked end 54/64/74 that provides pulling capabilities without needing to touch surfaces like door knobs, certain faucets, trash can lids, grocery bag handles, etc. Therefore, in the public restroom scenario above, the person finding themselves with an inwardly opening door is able to access their hand sanitizer dispensers 50/60/70 (e.g. from a pocket or handbag), engage the hooked end 54/64/74 with the door knob, pull open the door, then spray sanitizer on their hands, just in case.

In FIG. 2, the hand sanitizer dispenser 50 has a body portion 8/52/14/16 and optionally, a cap or cover portion 18/20. The body portion 8/52/14/16 has a generally hollow, often tubular reservoir 52 for holding a liquid (the hand sanitizer fluid), a bottom 8 that closes the reservoir 12, and a dispensing portion 14/16 that includes a feed tube 14. When the hand sanitizer dispenser 50 is held upright (the bottom 8 is downward towards earth), the hand sanitizer fluid settles near the bottom 8 and, when the spray 16 is operated, the hand sanitizer fluid is drawn up the feed tube 14 and sprayed/atomized into the air around where the spray 16 is aimed. Although a spray 16 is shown, any form of dispensing of the hand sanitizer from within the reservoir 12 is anticipated, including pouring, extruding, etc.

To reduce leakage and inadvertent activation of the spray 16, many such hand sanitizer dispensers 50 include a cover 18. Also, many covers 18 have a clip 20 for removably affixing the hand sanitizer dispenser 10 to an article of clothing, a handbag, etc.

The hand sanitizer dispenser 50 includes a hook portion 54 that is formed or affixed to the bottom 8. In such, for certain manufacturing situations, it is anticipated that existing tools and molds are used to produce the body portion 8/52/14/16 and the cap or cover portion 18/20, and new tools and molds are needed only for the hook portion 54. The hook portion 54 is used to facilitate opening of doors, cabinets, trash cans, lifting certain objects, etc.

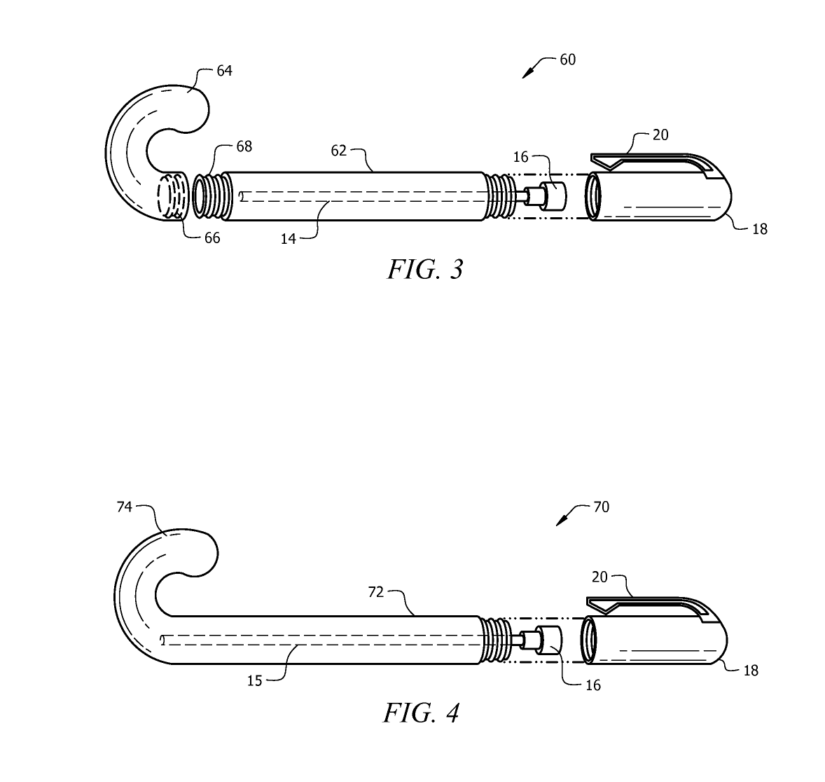

In FIG. 3, the hand sanitizer dispenser 60 has a body portion 8/62/14/16 and optionally, a cap or cover portion 18/20. The body portion 8/62/14/16 has a generally hollow, often tubular reservoir 62 for holding a liquid (the hand sanitizer fluid). A dispensing portion 14/16 includes a feed tube 14. When the hand sanitizer dispenser 60 is held upright, the hand sanitizer fluid settles near the bottom and, when the spray 16 is operated, the hand sanitizer fluid is drawn up the feed tube 14 and sprayed/atomized into the air around where the spray 16 is aimed.

To reduce leakage and inadvertent activation of the spray 16, many such hand sanitizer dispensers 60 include a cover 18. Also, many covers 18 have a clip 20 for removably affixing the hand sanitizer dispenser 10 to an article of clothing, a handbag, etc.

The hand sanitizer dispenser 60 includes a hook portion 64 that removably affixed to an end of the tubular reservoir 62. In such, the hook portion 64 is optionally added to the tubular reservoir 62 as needed. In some embodiments, the hook portion 64 also serves to cap and seal the tubular reservoir 62, providing access to the tubular reservoir 62 for refilling, etc. Additionally, as in the previous example of FIG. 2, the hook portion is manufactured with separate molds and tools, thereby providing manufacturing flexibility. As with the prior example, the hook portion 64 is used to facilitate opening of doors, cabinets, trash cans, and lifting certain objects, etc.

In FIG. 4, the hand sanitizer dispenser 70 has a body portion 8/72/14/16 and optionally, a cap or cover portion 18/20. The body portion 8/72/14/16 has a generally hollow, often tubular reservoir 72 for holding a liquid (the hand sanitizer fluid). A dispensing portion 14/16 includes a feed tube 14. When the hand sanitizer dispenser 70 is held upright, the hand sanitizer fluid settles near the bottom and, when the spray 16 is operated, the hand sanitizer fluid is drawn up the feed tube 14 and sprayed/atomized into the air around where the spray 16 is aimed.

To reduce leakage and inadvertent activation of the spray 16, many such hand sanitizer dispensers 70 include a cover 18. Also, many covers 18 have a clip 20 for removably affixing the hand sanitizer dispenser 10 to an article of clothing, a handbag, etc.

The hand sanitizer dispenser 70 includes a hook portion 64 that is formed as a continuation of the end of the tubular reservoir 72. In such, in some embodiments, the hook portion 74 provides further storage for the hand sanitizer fluid and, preferably, the feed tube 14 extends into the hook portion 74 to access the additional capacity provided by the hook portion 74. As with the prior example, the hook portion 74 is used to facilitate opening of doors, cabinets, trash cans, and lifting certain objects, etc.

Note that, although the hand sanitizer dispensers 50/60/70 are shown with a specific shape and dispensing configuration, there is no limitation on such and any shape and dispensing configuration is anticipated. For example, a substantially rectangular cross section is anticipated, a triangular cross section is anticipated, a “squeeze bottle” dispenser is anticipated, a “pour” dispenser is anticipated, any size or shape of hook is anticipated, hollow or solid, etc. In embodiments in which the dispensing system is, for example, a squeeze/extruding system or a pouring system, it is anticipated that a cap seal the dispensing end of the reservoir.

Also note that it is anticipated that the hook portions 54/64/74 are made from a similar or a different material than a material that is used to fabricate the body portions 8/52/62/72/14/16. It is anticipated that the hook portions 54/64/74 be made of a stiff material so as to retain their shape under the force of, for example, pulling a door open or lifting a bag of groceries.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

1. A hand sanitizer dispenser comprising:
a body portion having a substantially tubular cross-sectional shape, having a hooked end, and having a substantially straight body, the body portion having a reservoir within the hooked end and within the substantially straight body a sprayer interfaced to an end of the substantially straight body, the sprayer having a feed tube extending into the reservoir, whereas pressing on the sprayer extracts a portion of the liquid from the reservoir, through the feed tube, and emits the liquid;
whereas the hooked end is sized for pulling objects.

2. The hand sanitizer dispenser of claim 1, wherein the hooked end is made of a material that has sufficient rigidity as to enable pulling open a door.

3. The hand sanitizer dispenser of claim 1, further comprising a sealing cap that removably covers the sprayer, removably held over the sprayer by a snap fit.

4. The hand sanitizer dispenser of claim 1, wherein the feed tube extends linearly into the reservoir.

5. The hand sanitizer dispenser of claim 1, further comprising a cover, the cover removably engaged over the sprayer.

6. (canceled)

7. The hand sanitizer dispenser of claim 1, wherein the feed tube extends into the hooked end.

8. (canceled)

9. A method of maintaining hand sanitation, the method comprising:
providing a hand sanitizer dispenser with hook, the hand sanitizer dispenser comprising:
a body portion having a substantially tubular shape containing there within a reservoir for holding a liquid, the body portion having a tubular side and a first end connected to an end of the tubular side, an atomizer interfaced to the first end, the atomizer having a feed tube extending into the reservoir, whereas pressing on the atomizer extracts a portion of the liquid from the reservoir, through the feed tube, and sprays the liquid; and
a hook portion formed or affixed to a second end of the body portion, the second end opposing the first end, the reservoir extends into the hook portion;
holding the body portion in one hand;
engaging an object with the hook portion;
moving the object by way of the hook portion without making direct contact between the one hand and the object; and
releasing the object from the hook portion.

10. The method of claim 9, wherein the object is a door handle.

11. The method of claim 9, wherein the object is a grocery bag.

12. The method of claim 9, wherein the object is trash can lid.

13. The method of claim 9, wherein the object is a faucet.

14. The method of claim 9, wherein the object is a light switch.

15. The method of claim 9, wherein the object is an airplane door pull.

16. The method of claim 9, wherein the liquid is a hand sanitizer liquid.

17. A hand sanitizer dispenser comprising:
a hollow body portion having a hook-shaped end and a substantially linear end, thereby forming a reservoir throughout the entire body portion, a spraying device is interfaced to an end of the hollow body portion, the end being distal from the hook-shaped end, the spraying device having a feed tube extending into the reservoir to a location at least within the hook-shaped end, whereas pressing on the spraying device extracts a portion of the liquid from the reservoir, through the feed tube, and sprays the liquid; and a cover, the cover removably engaged to the first end of the body for covering of the atomizer;
whereas the hollow body portion is made of a material that is stiff enough as to enable opening of a door by pulling a handle of the door with the hook portion.

18. The hand sanitizer dispenser of claim 17, wherein the cover is held to the first end by a snap interface.

19. The hand sanitizer dispenser of claim 17, wherein the reservoir contains a liquid.

20. The hand sanitizer dispenser of claim 19, wherein the liquid is a hand sanitizer.
